(19) United States
(12) Patent Application Publication
(10) Pub. No.: US 2026/0182929 A1
(43) Pub. Date: Jul. 2, 2026

(54) COLORECTAL CANCER RISK ASSESSMENT

(71) Applicants: Denis King, Sylvania (AU); Yaarob Khamas, Milsons Point (AU)

(72) Inventors: Denis King, Sylvania (AU); Yaarob Khamas, Milsons Point (AU)

(21) Appl. No.: 19/132,038

(22) PCT Filed: Nov. 24, 2023
(86) PCT No.: PCT/AU2023/051210

§ 371 Date: May 22, 2025

(30) Foreign Application Priority Data
Nov. 25, 2022 (AU) 2022903574

(51) Int. Cl. A61B 5/00; G16H 10/60; G16H 50/30

(52) U.S. Cl. CPC A61B 5/7275; G16H 10/60; G16H 50/30

(57) ABSTRACT

This disclosure relates to a method for detecting colorectal cancer. The method comprises determining whether an advanced neoplasm is present in a first screening time period and upon determining that an advanced neoplasm is present in the first screening time period, performing colonoscopy in a second screening time period, after the first screening time period, to detect colorectal cancer. There is further provided a method for stratification of a subject comprising upon detection of an advanced neoplasm in a first screening time period, stratifying the subject into a high risk stratification for developing colorectal cancer in a second screening time period. There is further provided a computer implemented method for calculating a risk score for developing colorectal cancer comprising determining patient data; applying a trained machine learning model to the patient data to determine the risk score; and outputting the risk score.

### CROSS-REFERENCE TO RELATED APPLICATIONS

The present application claims priority from Australian Provisional Patent Application No 2022903574 filed on 25 Nov. 2022, the contents of which are incorporated herein by reference in their entirety.

### TECHNICAL FIELD

This disclosure relates to detecting colorectal cancer and to assessing the risk for colorectal cancer.

### BACKGROUND

Colorectal cancer is the third most commonly diagnosed cancer worldwide after lung and breast and is responsible for the second highest number of cancer deaths. A key strategy in decreasing the incidence of colorectal cancer (CRC) is colonoscopic screening and removal of premalignant lesions

When counselling patients about screening high risk groups, clinicians have generally have to offer advice on the basis of the relative risk of a group of people with similar risk factors. Patients enter a screening program according to their perceived risk and then continue in that program for up to 25 years or more, based on the primary indication and, to some degree, irrespective of interim findings.

Existing guidelines on using colonoscopy to screen or conduct surveillance on people at high risk of developing colorectal cancer have a reasonable level of agreement on surveillance intervals, with variations around the definitions used to determine high risk and the ages at which initial screening or surveillance should start. However, the evidence supporting these surveillance intervals is either of poor or very low quality, with only the occasional reference to evidence being of moderate quality. Most of the guidelines recommend screening between 50 and 75 years of age with either colonoscopy (every ten years, but for some indications, as frequently as every three to five years), flexible sigmoidoscopy (every five years) or a faecal occult blood test (annually or biennially). What is currently lacking is an evidence-based stratification mechanism to determine which individuals, among the high-risk groups, are actually at increased risk.

At present there are no known biochemical or genetic markers that may accurately predict whom among such a group actually is at risk during the screening period.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SUMMARY

A method for detecting colorectal cancer in a subject comprises:
determining whether an advanced neoplasm is present in a first screening time period;
upon determining that an advanced neoplasm is present in the first screening time period, performing colonoscopy in a second screening time period, after the first screening time period, to detect colorectal cancer.

In some embodiments, the method further comprises upon determining that no advanced neoplasm is present in the first screening time period, performing community surveillance in the second screening time period.

In some embodiments, detection of no advanced neoplasm comprises detection of non-significant adenomas and no detection of advanced neoplasms.

In some embodiments, the first screening time period is 10 years.

A method for stratification of a subject comprises upon detection of an advanced neoplasm in a first screening time period, stratifying the subject into a high risk stratification for developing colorectal cancer in a second screening time period.

In some embodiments, the method further comprises upon detection of no advanced neoplasm in the first screening time period, stratifying the subject into a low risk stratification for developing colorectal cancer in the second screening time period.

In some embodiments, detection of no advanced neoplasm comprises detection of non-significant adenomas and no detection of advanced neoplasms.

A computer implemented method for calculating a risk score for developing colorectal cancer comprises:
determining patient data indicative of patient medical history and patient information;
applying a trained machine learning model to the patient data to determine the risk score;
outputting the risk score.

In some embodiments, the patient medical history comprises results from a colonoscopy in a first screening time period.

In some embodiments, the results from the colonoscopy is indicative of advanced neoplasia in the first screening time period.

In some embodiments, the method further comprises selectively indicating a colonoscopy in a second screening time period based on the risk score.

In some embodiments, determining the patient data comprises extracting the patient data from patient medical records.

In some embodiments, determining the patient data comprises extracting the patient data from patient management software.

In some embodiments, the patient medical records are indicative of a history of polyps of types 1 to 5 in the first screening time period.

In some embodiments, the patient medical records further comprise one or more of:
demographics;
medical conditions other than colorectal cancer or polyps; and
medical imaging data.

In some embodiments, the trained machine learning model is a non-linear algorithm.

In some embodiments, the non-linear algorithm comprises one or more of:
a classification and regression tree;
a naive Bayes method;
a K-Nearest Neighbours method;
a Learning Vector Quantization method; or
a Support Vector Machine.

In some embodiments, the method further comprises training a machine learning model on historical patient data to obtain the trained machine learning model.

In some embodiments, the training comprises adjusting parameters of the machine learning model to reduce an error between (i) an output of the machine learning model and (ii) historical patient data indicative of a presence or absence of colorectal cancer associated with the historical patient data.

In some embodiments, the training comprises:
identifying clusters in the historical patient data; and
fitting the clusters to defining data points.

In some embodiments, the defining data points comprise a type of polyp detected in the first screening time period.

In some embodiments, the method further comprises:
determining a treatment plan based on the risk score; and
managing the patient by implementing the treatment plan.

In some embodiments, determining the treatment plan comprises determining a schedule of colonoscopy procedures based on the risk score.

In some embodiments, the patient medical data comprises data relating to lifestyle factors.

### BRIEF DESCRIPTION OF DRAWINGS

FIG. 1 illustrates the survival curve for first occurrence of significant polyps or CRC, stratified by findings in first 10 years. Total cohort (5006 patients: 72,081 years of follow-up): any period of follow-up: Occurrence of AN recorded only once for each patient, that is number of subjects affected, but no assessment of total AN load.

FIG. 2 illustrates, for the study group the survival curve (lifetable method) for first occurrence of advanced neoplasia by year since presentation, stratified by findings in first 10 years (n=2,178): 10 years of follow-up: Number of subjects affected, but no assessment of total AN load.

FIG. 3 illustrates, for the study group, the total number of advanced neoplasia per person per year, by findings in first 10 years (normal or non-advanced adenoma (bottom line) vs advanced neoplasia (top line)) (n=2,178): >10 yr follow-up: total load of AN per subject.

FIG. 4 illustrates a method for detecting colorectal cancer.

FIG. 5 illustrates a computer system for detecting colorectal cancer.

### DESCRIPTION OF EMBODIMENTS

There are two main molecular pathways described for the development of CRC. The first, responsible for the majority of cancers, is the standard adenoma-cancer pathway seen in the general population, as well as in high-risk groups such as familial adenomatous polyposis. This pathway demonstrates a greater propensity to left-sided carcinoma. The alternative pathway, only fully recognised and studied in the last 20-30 years, is the serrated pathway. This pathway exhibits different genetic abnormalities, is estimated to account for up to 30% of CRC and has a propensity for the right colon. For almost all CRC there is a predictable pathway from normal tissue, through advanced neoplasia (AN), to cancer.

This disclosure provides data from a longitudinal study of 5,006 consecutive individual patients referred for colonoscopic screening because of a perceived high risk of colorectal cancer, followed up for a total of 72,082 patient years. The data that informed this study were collected prospectively. Of the 5,006 patients, 2,178 (43.5%) were followed for more than ten years [total patient years: 37,633; range: 10-1¬-41 yr; mean: 17.24 yr (IQR 12.57-20.73)].

The results of this study suggest that it is possible to identify the features of individuals within a sub-group in the first ten years of the recommended screening period who have an increased likelihood of developing premalignant lesions for at least the following 15 years.

Having identified the patients within the overall at-risk cohort who are actually at increased risk, it may be possible to strengthen the evidence for them remaining in a surveillance program. For those who do not appear to be at increased risk, which may be as much as 20% of the total cohort, it may be possible to continue surveillance using standard community protocols, such as faecal testing, rather than intrusive protocols involving colonoscopy. This would reduce the attendant procedural morbidity and mortality, and cost to the community, without an adverse impact upon screening efficacy.

This study disclosed herein has identified patient characteristics within the first ten years of the recommended screening period that predict the likelihood of developing premalignant lesions over the following 15 years.

Patients with advanced neoplasia (AN) in the first ten years of screening were found to have a significantly higher risk of subsequently developing AN when compared to those with no AN. This effect continued at least for the standard surveillance timeframe, currently a total of 25 years.

### Definitions

An adenoma is a benign tumor (that is not a cancer) of epithelial tissue with glandular origin, glandular characteristics, or both. Neoplastic polyps that are benign are referred to as adenomas.

A polyp is an abnormal growth of tissue in the colon. Polyps are either pedunculated (attached to the intestinal wall by a stalk) or sessile (grow directly from the wall).

A hyperplastic polyp is a polyp that has no malignant potential and is therefore no more likely than normal tissue to eventually become a cancer.

A neoplasm is a tissue whose cells have lost normal differentiation. They can be either benign growths or malignant growths. The Haggitt's criteria has level 0 through level 4, with all invasive carcinoma of sessile polyp variant by definition being classified as level 4. Level 0: Cancer does not penetrate through the muscularis mucosa. Level 1: Cancer penetrates through the muscularis mucosa and invades the submucosa below but is limited to the head of the polyp. Level 2: Cancer invades through with involvement of the neck of polyp. Level 3: Cancer invades through with involvement of any parts of the stalk. Level 4: Cancer invades through the submucosa below the stalk of the polyp but above the muscularis propria of the bowel wall.

Advanced neoplasia (AN) is the presence of a neoplasm with lesions larger than 10 mm with a villous content or with high-grade dysplasia or carcinoma. AN may be defined or diagnosed by the protocol or features described in Imperiale et al. (N Engl J Med 2014; 370:1287-97), which is incorporated by reference herein.

### Methods

This data disclosed herein is from a study that is a retrospective review of prospectively collected data over a 43-year period in a single colorectal surgical practice in Sydney, Australia. The Human Research Ethics Committee of the University of Wollongong, Australia (approval 2020/386) approved the project in 2020, after the completion of recruitment.

### Patient Inclusion Criteria

All patients in the study cohort were treated at some time during their period of surveillance.

All patients with the defined risk factors presenting to the practice were included, other than those listed in the exclusion criteria. Study patients were identified as being at high risk of developing CRC due to a significant family history of CRC (FHCC), previous removal of adenomatous polyps or previous surgical resection of CRC, on the basis of the NHMRC guidelines.

In cases of ‘no findings’ at index colonoscopy, for example with FHCC, the follow-up period commenced from the date of index colonoscopy, rather than the date that would have been recommended by the NHMRC for the commencement of screening, reflecting normal clinical practice. That aspect is the subject of further study. For patients presenting with neoplasia, the follow-up period commenced from the date the colon was cleared of any neoplasia. Multiple colonoscopies for completion polypectomies were counted as one, with the follow-up starting from the initial colonoscopy, as the completion colonoscopies are designed to deal with those polyps that are found but not removed at the original colonoscopy.

TABLE 1
Patient categorisation by high-risk category

| | Category | Definition |
|---|---|---|
| Family | FH1 | Single relative 55 years of |
| History CRC | (x2 risk) | age or older. |
| | FH2 | One 1^{st} degree relative <55 years of age. |
| | (x3-6 risk) | Two 1^{st} degree relatives at any age. |
| | | One 1^{st} degree and two 2^{nd} degree |
| | | relatives at any age. |
| | FH3 | Three 1^{st} or 2^{nd} degree relatives, |
| | (x7-10 risk) | one <55 years of age. |
| | | Three 1^{st} degree relatives at any age. |
| | FH4* | Familial adenomatous polyposis (FAP), |
| | | Lynch and related syndromes. |
| | FH5^{†} | Details incomplete. |
| Cancer | CA1 | Single cancer with <5 adenomatous |
| Follow-up | | polyps. |
| | CA2 | Multiple cancers. |
| | CA3 | Single cancer with significant polyps. |
| | CA4^{‡} | Details incomplete. |
| Polyp | P1 | Hyperplastic polyps. |
| Follow-up | P2 | Adenomatous polyps <10 mm. |
| | P3 | Adenomatous polyps ≥10 mm. |
| | P4 | Tubulovillous adenoma (TVA) or |
| | | sessile serrated adenoma (SSA). |
| | P5^{§} | Details incomplete. |
| Procedure | A1 | Poor preparation. |
| incomplete | A2 | Medical reasons. |
| | A3 | Sigmoid stricture. |
| | A4 | Perforation. |

*FH4 relates to conditions considered to be transmitted by a specific gene, usually autosomal dominant, with penetration of near 100% with FAP and approximately 80% with Lynch and related syndromes. Such conditions make up perhaps 3% of colorectal cancers and have been excluded from consideration in this study. The reason for that is that genetic testing is now available and therefore it is possible to determine those at risk and once that is done such people warrant intensive follow-up along well-established guidelines.
^{†}FH5 - relatives are known to have CRC but details such as age of onset are unknown.
^{‡}CA4 - patients are known to have CRC but details such as age and sometimes associated pathology are unknown.
^{§}P5 - the polyp is adenomatous by nature, but all details of numbers and size are not present.

The patients were categorised into risk factors, with allowances for some with incomplete information. All patients were referred from a general practitioner/family doctor and had a formal consultation prior to the initial procedure. The reason for the patient's referral was discussed, a history was sought regarding previous colonic problems and pathology, family history of CRC, and previous colonic investigations including colonoscopy. At the first visit patients gave consent for the collection and use of clinical information consistent with this study.

Polyp size up to 10 mm was visually assessed before polypectomy by comparison with biopsy forceps (7 mm when fully opened). Polyps greater than 10 mm were retrieved and measured directly as part of standard histopathological assessment which has been demonstrated to be superior to endoscopic estimates of size. All histology was performed by pathologists with a particular interest in colorectal pathology. Any uncertainty regarding the nature of any specimen was discussed in a formal Sydney Colorectal Associates Unit meeting with a panel of up to four colorectal histopathologists.

The complete cohort was assessed, and then for further clarification, the subset of patients who had been followed for more than ten years was studied to assess the long-term validity of the findings, and possible mechanisms.

### Patient Exclusion Criteria

Patients were excluded from the study based on three criteria:
(1) presence of family cancer syndromes, such as Familial Adenomatous Polyposis or Hereditary Nonpolyposis Colorectal Cancer, latterly on the basis of genetic testing;
(2) those perceived to have an added independent risk due to the presence of ulcerative colitis; and
(3) those with a history of pelvic radiotherapy for unrelated conditions.

### Study Data Set

The database of patient information was established in 1977 for quality assurance purposes and clinical research projects. The surgical practice conducted a comprehensive recall program involving written reminders, phone calls from nursing staff, and then contact with referring doctors.

The results of all investigations, including procedure reports, measurement of specimens, and histology reports, were entered upon receipt, usually within five days of the procedure. The database was privately developed in 1976 and converted in January 2000 to a FoxPro database. The database is now housed by Amazon Cloud Web Services (Amazon, Seattle, USA), with regular back-ups.

### Statistical Analysis

De-identified information from the database was exported into a Microsoft Excel (Microsoft, Seattle, USA) spreadsheet for analysis and coded by high-risk category. The data were checked against the original clinical records to ensure accuracy and completeness.

TABLE 2
Stratification criteria

| | |
|---|---|
| Non-significant | P1 - Hyperplastic polyps. |
| adenomas (NSA) | P2 - Adenomatous polyps <10 mm. |
| Advanced | P3 - Adenomatous polyps ≥10 mm. |
| neoplasia (AN) | P4 - Tubulovillous adenoma (TVA) or |
| | sessile serrated adenoma (SSA). |
| | CA1 - Single cancer with <adenomatous |
| | polyps. |
| | CA2 - Multiple cancers. |
| | CA3 - Single cancer with significant |
| | polyps. |
| | CA4 - Details incomplete. |

In the analyses, the grouping was influenced by the evidence that individuals with no significant adenomas have no significantly increased risk of CRC compared with the general population. The study cohort therefore was categorised into two groups, no significant adenomas (NSA), and advanced neoplasia (AN), based on findings in the first ten years of surveillance.

The data for each group were analysed from year 11 to year 25 after index colonoscopy. Incidence rates were calculated for each group as the number of occurrences of AN, or as the total load of AN, as a proportion of the number of patients remaining in that group for each year since index colonoscopy.

Survival analysis was performed using the year post-index colonoscopy as the ‘time-to-event’ variable. Analyses were stratified by findings in the first ten years (N, NAA, AN). Patients were included in analyses until their last colonoscopy.

Results were presented as survival curves using the life table (actuarial) estimate of the distribution, with 95% confidence intervals. Life table estimates were chosen over Kaplan-Meier as the actual date of the occurrence of a polyp was not known, rather the date they were observed (i.e. during their next colonoscopy). Survival curves were compared using the log-rank test and the null hypothesis rejected (p<0.0001). Data were analysed using SAS version 9.4 and extracted into Microsoft Excel for graphing.

### Results

Recruitment of the patient cohort started in 1977 and finished in 2018. Patients were followed up until contact was lost, or until 31 Dec. 2020. The total patient cohort comprised 5,006 consecutive patients, with 72,082 patient-years of follow-up.

Approximately 90% of colonoscopies were performed, with the remainder performed by medical practitioners who were being trained or had been trained in colonoscopy by the lead author, or occasionally by other accredited specialist colonoscopists.

For the purposes of this study, 2,178 patients with more than ten years of follow-up were included. This cohort had undergone 12,167 colonoscopies (mean—5.59), with 37,597 total patient-years of follow-up (mean—17.26 years; IQR 12.57-20.73).

TABLE 3
Characteristics of study cohort at index colonoscopy

| | | | | Previous | |
|---|---|---|---|---|---|
| | | | Significant | removal of | Previous |
| | | | family | significant | surgical |
| | | | history | colorectal | resection |
| | | Whole | of CRC | polyps | of CRC |
| | | cohort | (n = 1175) | (n = 708) | (n = 394) |
| | | (n = 2178) | FH1-FH5 | P1-P5 | Ca1-Ca4 |
| Demographics | | | | | |
| Sex | Male | 1122 (51.52%) | 520 (44.26%) | 439 (62.00%) | 212 (53.81%) |
| | Female | 1056 (48.48%) | 655 (55.74%) | 269 (38.00%) | 182 (46.19%) |
| Age (years) | | | | | |
| <50 | Male | 354 (16.25%) | 240 (20.43%) | 97 (13.70%) | 32 (8.12%) |
| | Female | 357 (16.39%) | 288 (24.51%) | 55 (7.77%) | 27 (6.85%) |
| 50-54 | Male | 160 (7.35%) | 84 (7.15%) | 62 (8.76%) | 18 (4.57%) |
| | Female | 142 (6.52%) | 94 (8.00%) | 34 (4.80%) | 18 (4.57%) |
| 55-59 | Male | 178 (8.17%) | 65 (5.53%) | 75 (10.59%) | 44 (11.17%) |
| | Female | 162 (7.44%) | 92 (7.83%) | 45 (6.36%) | 31 (7.87%) |
| 60-64 | Male | 152 (6.98%) | 52 (4.43%) | 71 (10.03%) | 37 (9.39%) |
| | Female | 152 (6.98%) | 78 (6.64%) | 47 (6.63%) | 36 (9.14%) |
| 65-69 | Male | 172 (7.9%) | 49 (4.17%) | 86 (12.15%) | 45 (11.42%) |
| | Female | 156 (7.16%) | 72 (6.13%) | 51 (7.20%) | 45 (11.42%) |
| 70-74 | Male | 76 (3.49%) | 26 (2.21%) | 37 (5.23%) | 21 (5.33%) |
| | Female | 59 (2.71%) | 21 (1.79%) | 27 (3.81%) | 15 (3.81%) |
| >75 | Male | 30 (1.38%) | 4 (0.34%) | 11 (1.55%) | 15 (3.81%) |
| | Female | 28 (1.29%) | 10 (0.85%) | 10 (1.41%) | 10 (2.54%) |

Ninety nine percent of the examinations were completed to the caecum or an obstructing lesion. The majority of failures were due to poor preparation. There were eight perforations, mostly related to removal of large sessile polyps and requiring surgery, and no deaths.

TABLE 4
Colonoscopy quality parameters

| | | % of | Fe- | % of | | % of |
|---|---|---|---|---|---|---|
| | Male | total | male | total | Total | total |
| Patients | 1122 | 51.51% | 1056 | 48.48% | 2178 | |
| Colonos- | 6338 | 52.09% | 5828 | 47.90% | 12166 | |
| copies | | | | | | |

Adenomas

| | | | | | | |
|---|---|---|---|---|---|---|
| Adenoma | 1453 | 22.93% | 892 | 15.30% | 2345 | 19.28% |
| (P2- | | | | | | |
| CA4) | | | | | | |

Complications

| | | | | | | |
|---|---|---|---|---|---|---|
| Incom - | 52 | 0.82% | 78 | 1.34% | 130 | 1.07% |
| plete | | | | | | |
| (A1-A5) | | | | | | |
| A1 - | 39 | 0.62% | 35 | 0.60% | 74 | 0.61% |
| Poor | | | | | | |
| prep | | | | | | |
| A2 - | 1 | 0.02% | 2 | 0.03% | 3 | 0.02% |
| Medical | | | | | | |
| A3 - | 9 | 0.14% | 35 | 0.60% | 44 | 0.36% |
| Stricture | | | | | | |
| A4 - | 3 | 0.05% | 5 | 0.09% | 8 | 0.07% |
| Perfor- | | | | | | |
| ation | | | | | | |
| A5 - | 0 | 0.00% | 1 | 0.02% | 1 | 0.01% |
| Equip- | | | | | | |
| ment | | | | | | |
| Failure | | | | | | |

Table 4 also outlines the adenoma detection rate (ADR) in this study. All of those in this study had the colon cleared of polyps before entering surveillance.

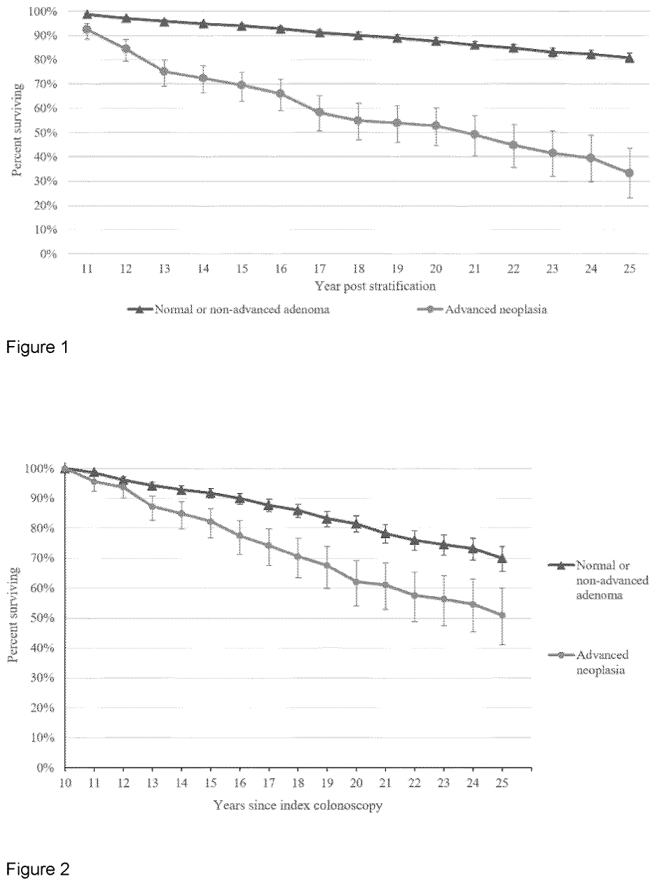

An initial assessment the entire cohort of 5006 was assessed. The results are shown in FIG. 1. This showed that there is a significant difference between the subjects with AN in the first ten years, and those without, and that the difference in trajectory continued for a total of 15 years, to 25 years in total.

An assessment of the study group of 2,178 patients (Table 3 and FIG. 2) shows a significant difference in years 11 and 25, between those with no significant findings in the first ten years, and the remainder. This is manifest by the greater likelihood of individual patients developing at least one AN in that period.

The total load of AN per patient is an indicator of risk of CRC. An assessment of total AN load in the study group shows a disparity between those with AN in the first ten years, and those without. As well as the greater numbers developing AN, the rate at which AN occurs, and therefore the premalignant load, is greater in the first ten years, and continues for the whole period studied.

In this study patients were stratified into two groups on the basis of colonoscopic findings in the first ten years of follow-up. The first incidence of an advanced neoplasm in years 11-25, stratified by results in the first ten years of surveillance, was determined. Each patient is recorded only once, irrespective of the number of advanced neoplasms that were subsequently found. (FIG. 1)

In the entire group, there was a difference between the cohort trajectories from year 11 to 25.

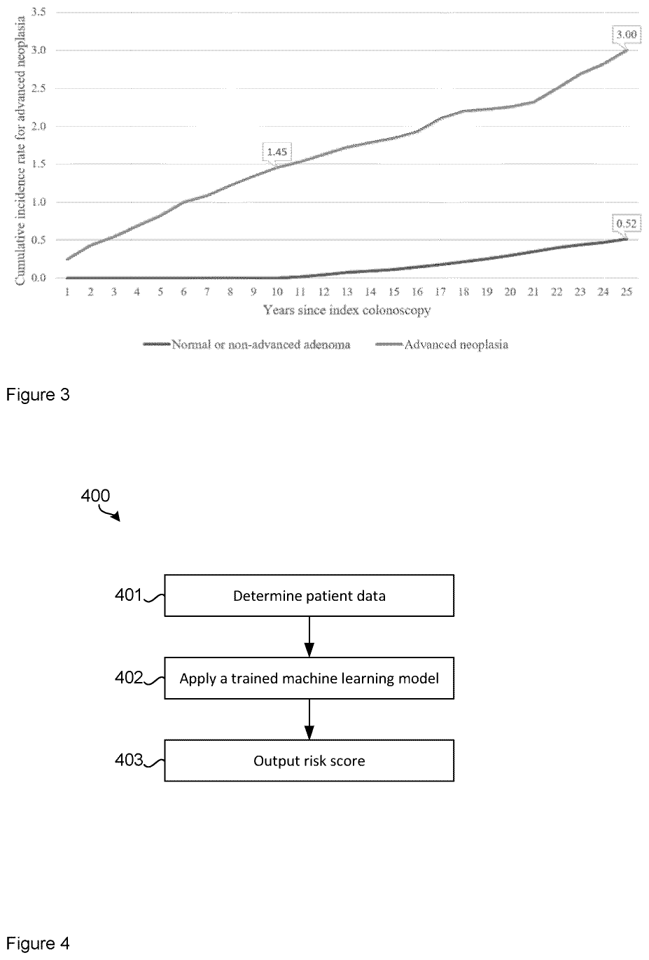

It would appear that the total load of advanced neoplasia is a better determinant of CRC risk. The cumulative numbers of advanced neoplasms in each of the three groups was established with the count starting from the beginning of surveillance. (FIG. 3) This graph also shows a diverging trajectory continuing from years 11-25.

The findings indicate that those patients who have normal or small hyperplastic polyps, or non-advanced adenomas in the first ten years have a reduced risk of developing advanced neoplasia compared with those who develop advanced neoplasia in the first ten years.

Therefore, patients who have normal or small hyperplastic polyps, or non-advanced adenomas in the first ten years may be managed effectively with secondary community screening based on faecal occult blood testing, with the corresponding lessening of inconvenience, morbidity, and societal cost.

Those who appear to have a high risk of developing AN may be counselled about the need for continuing intensive surveillance on the basis of their own medical history, rather than that of an overall group to which they belong.

Stratification may mean that those who are become aware that they are at proven risk may become more compliant with screening programs, which have traditionally been compromised by low compliance rates.

With the possibility of more accurate stratification methods, the cost-effectiveness of screening colonoscopy could be enhanced.

### Strengths

The major strength of this study is the extended period of surveillance follow-up after index colonoscopy, using prospectively collected data. Selection bias was minimised by treating every patient presenting with the required entry criteria, as identified from national guidelines. One significant pathology parameter was the presence or otherwise of adenomatous polyps. This was very clear in the vast majority of cases. In case of any doubt, pathology findings were agreed by expert consensus. The study was not subject to incidence-prevalence bias.

### CONCLUSION

This disclosure provides a stratification method within the first ten years of surveillance of people believed to be at increased risk of developing CRC that appears to remain valid for at least 25 years. This may guide long-term surveillance by quantifying the likely propensity to the development of CRC for individual patients. This may allow practitioners to better counsel patients about the options for CRC surveillance, including use of community-based secondary screening for many.

Accordingly, this disclosure provides a method for detecting colorectal cancer in a subject. The method comprises determining whether an advanced neoplasm is present in a first screening time period. This first screening period may be 10 years as set out in the examples above. However, other time periods are equally possible. For example, first screening period may be 5 years or 6, 7, 8, or 9 years. The first screening period may also be 11, 12, 13, 14, 15, 16, 17, 18 or 20 years.

Upon determining that an advanced neoplasm is present in the first screening time period, the disclosed method comprises performing colonoscopy in a second screening time period, after the first screening time period, to detect colorectal cancer. The second time period may be 25 years. The second time period typically starts immediately after the first time period but there may also be a gap between the first time period and the second time period. The second time period may also be any of 10, 11, 12, 13, 14, 15, 16, 17, 18, 19, 20, 21, 22, 23, 24, 25, 26, 27, 28, 29, or 30 years.

In other words, upon detection of an advanced neoplasm in the first screening time period, the subject is stratified into a high risk stratification for developing colorectal cancer in the second screening time period.

### Machine Learning Method

The description above provides a stratification for a given patient into risk categories. This stratification can be implemented as a classifier in computer program code. It is noted that the data from the study above was approached by the data scientist without knowing what clinical hunch the data owner had, or what their observations over the years would say about it.

To that end, FIG. 4 illustrates a computer implemented method 400 for calculating a risk score for developing colorectal cancer. The method comprises determining 401 patient data indicative of patient medical history and patient information. The patient medical history comprises results from a colonoscopy in the first screening time period. As set out above, the results from the colonoscopy is indicative of advanced neoplasia in the first screening time period.

It is noted that determining the patient data may comprise extracting the patient data from patient medical records, such as by extracting the patient data from patient management software, also referred to as electronic medical record (EMR).

The patient medical records are indicative of a history of polyps of types 1 to 5 in the first screening time period, which can be used as features of the model. In this sense, the features listed in Tables 1 and 2 may also be features of the model. The patient medical records used in the model may further comprise demographics, medical conditions other than colorectal cancer or polyps, or medical imaging data, such as X-ray, computed tomography, ultrasound, magnetic resonance imaging or other medical imaging.

Once the patient data is determined (or extracted), the method 400 applies 402 a trained machine learning model to the patient data to determine the risk score. The machine learning model is trained on the historical patient data of the study described above to obtain the trained machine learning model. The training is performed by adjusting parameters of the machine learning model to reduce an error between (i) an output of the machine learning model and (ii) historical patient data indicative of a presence or absence of colorectal cancer associated with the historical patient data. For example, in a convolutional neural network, the neural network parameters, such as convolutional filters, are adjusted to reduce the error. This can involve the use of back propagation and gradient descent methods.

The training may also comprise identifying clusters in the historical patient data and fitting the clusters to defining data points, such as a type of polyp detected in the first screening time period.

In one example, the data points used as features to the machine learning model comprise:
History of polyps 1-5 in the first 10 years of medical specialist check ups
Demographics (age, sex, postcode, employment/relationship status etc)
Other medical conditions
Potential imaging/ultrasound data

For example, Patient 1 has seen a physician for 10 years, their data will be uploaded to the algorithm API. They will then get a score:
Risk of cancer 1 in the next 10 years: 80/100
Risk of cancer 2 in the next 10 years: 20/100

The machine learning model may comprise a non-linear algorithm, such as a classification and regression tree; a naive Bayes method; a K-Nearest Neighbours method; a Learning Vector Quantization method; or a Support Vector Machine.

Finally, the method 400 outputs 403 the risk score. This may occur in a variety of different forms, such as in a patient report, on a computer display, such as on a screen or a mobile phone application. In other examples, the outputting means the writing of the risk score into an electronic medical record associated with the patient. Method 400 may also generate an alert or a automatically generated message that can be send to the practitioner or the patient.

Based on the calculated risk score the method may further selectively indicating a colonoscopy in a second screening time period. That is, in response to determining a risk score that is above a predefined threshold, the method generates an indication that a colonoscopy is required. The method may automatically contact a patient, such as via email and SMS. The method may also suggest a number of available time slots for performing the colonoscopy at a particular location and automatically book the colonoscopy based on a response from the patient.

Further, the method 400 may comprise determining a treatment plan based on the risk score and then managing the patient by implementing the treatment plan. The treatment plan may comprise determining a schedule of colonoscopy procedures based on the risk score. Depending on the findings of the scheduled colonoscopies, any ANs or other polyps are removed.

### Computer System

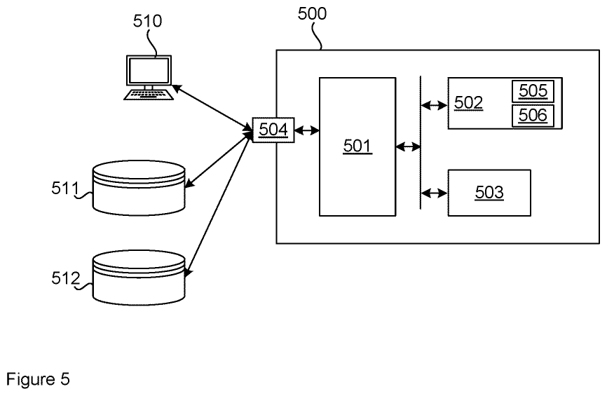

FIG. 5 illustrates a computer system 500 comprising a processor 501, program memory 502, data memory 503 and a communication port 504. The program memory 502 is a non-volatile computer-readable medium that stores program code. The program code implements the methods disclosed herein, such as method 400 in FIG. 4. That is, program memory 502 stores a data extraction module 505 that extracts patient information from patient data and a machine learning module 506 that applies a trained machine learning model to the patient information. More particularly, the patient information is the input to the machine learning model and the risk score is the output of the machine learning model. The program code, i.e. software, when executed by processor 601, causes the processor to perform the methods disclosed herein, such as method 400 in FIG. 4.

The communication port 504 is configured to communicate with a user terminal 510, such as a computer of a clinical practitioner, to receive the patient information. Communication port 504 may also be configured to communicate with a patient database 511 that hosts electronic medical records of patients. Through this connection, processor 501 receives the patient information and can extract it. Processor 501 may also write-back the risk score or other calculated outputs, including a recommendation for colonoscopy, risk score or the patient management plan, onto the user terminal 510 or the patient database 511.

Communication port 504 is further configured to communicate with a training database 512 that stores historical patient information. Processor 501 uses this information to train the machine learning model in machine learning module 506. This training may be performed once or may be performed or refined multiple times as more patient training data becomes available.

It is noted that computer system 500 may be implemented on a single dedicated computer, in a distributed computing environment, such as Amazon Web Services (AWS), or other cloud computing platform. Computer system 500 may further provide a web-service that provides the functionality of method 400 through an application programming interface or otherwise over the internet.

### Further Factors

In further examples, the risk stratification comprises calculating an odds ratio (OR) for each patient using the methods discussed above. The calculation of the OR may also incorporate environmental and lifestyle factors such as weight (expressed as Body Mass Index (BMI), a product of height and weight), family history of colorectal cancer, age, sex and smoking history (smoking for 30 yr or more—the effect dissipates within 10 years of cessation of smoking). The combination of those factors will allow an accurate assessment of the overall OR of developing colorectal cancer (CRC) on a personal rather than a group risk basis. In particular, the OR may be calculated by a regression model or other computer implemented tool.

As such, the combination of factors makes this application especially useful because the disclosed AI programme has the capacity to translate written clinical report data into the algorithm. The significance of the clinical component is in the development of a quantitative risk stratification mechanism that can predict an individual risk of the development of colorectal cancer (CRC). This will allow the separation of people within large groups believed to be at risk of CRC, such as those with a family history of the disease, into those that are at risk and those that are not. It will be appropriate for an AI-based program, and also allow that program to continually assess the clinical parameters, all being quantitative, to ensure the optimum validity of the measures used.

This will relieve those that have no personal risk of the burden of repeated colonoscopies over decades. It will also enhance the information available to those within the high-risk groups that actually are at risk, thus improving compliance, which is notoriously low in such screening programmes, partially because of the nature of the procedures.

This disclosure provides a stratification mechanism for an AI algorithm, not on the basis of modelling and conjecture, but on the basis of a 45-year study of more than 5000 patients perceived to be at high risk of CRC. This has produced an evidence based quantitative stratification method.

The fact that both of the critical elements in determining personal risk of CRC are available and suitable for analysis by AI, means that the development of a personal CRC risk assessment is achievable. As a result, it is now possible to change the way in which the screening of high-risk groups, that is targeted screening, is approached.

The first step is to determine the risk profile of an individual patient. Below is a data set as an example of demographic odds ratios.

Hippisley-Cox J, et al. BMJ Open 2015; 5: e007825. doi: 10.1136/bmjopen-2015-007825 Adjusted HRs with 95% CIs for cancers which occur in men and women in the derivation cohort
Smoking status: Heavy smoker (20+/day) Female: 1.17 (1.06 to 1.30) Male: 1.13 (1.05 to 1.22)
Alcohol: Very heavy drinker (>9 units/day) Female: 1.36 (0.80 to 2.32) Male: 1.56 (1.33 to 1.83)
DM2: Female: 1.16 (1.07 to 1.26) Male: 1.27 (1.20 to 1.35)

Family history predisposition can also be expressed as risk ratios. Risk ratios and odds ratios are calculated slightly differently, an odds ratio is generally a little higher than risk ratio, but they broadly address the same concept.

Examples of colorectal cancer (CRC) family history constellations and corresponding familial relative risk estimates-Taylor et al, Genetics in Medicine, 2011

| No. affected | No. affected | No. affected | Familial |
|---|---|---|---|
| first-degree | second-degree | third-degree | relative risk |
| relatives | relatives | relatives | (95% CI) |
| 0 | 0 | 0 | 0.83 (0.81-0.86) |
| 0 | 0 | ≥3 | 1.08 (0.97-1.20) |
| 0 | 1 | 2 | 1.33 (1.13-1.55) |
| 1 | 0 | 0 | 1.76 (1.63-1.89) |
| 1 | 0 | ≥3 | 2.01 (1.61-2.47) |
| 1 | 1 | 0 | 1.88 (1.59-2.20) |
| 1 | 1 | ≥3 | 3.28 (2.44-4.31) |
| 2 | 0 | 0 | 2.96 (2.41-3.60) |
| 2 | 0 | ≥3 | 4.82 (3.18-7.02) |
| 2 | 1 | 1 | 1.80 (0.82-3.41) |
| 2 | 1 | ≥3 | 4.67 (2.72-7.47) |
| ≥3 | 0 | 0 | 2.96 (1.42-5.44) |
| ≥3 | 0 | 1 | 4.21 (1.82-8.30) |
| ≥3 | 0 | ≥3 | 9.63 (5.26-16.15) |
| ≥3 | 1 | 0 | 12.39 (7.08-20.12) |
| ≥1 (dx | NA | NA | 3.31 (2.79-3.89) |
| age <50 yr) | | | |
| ≥1 (dx | NA | NA | 2.53 (2.24-2.85) |
| age 50-59 yr) | | | |
| ≥1 (fx | NA | NA | 2.22 (2.04-2.40) |
| age 60-69 yr) | | | |

The Australian government has determined that targeted screening is appropriate for those regarded as having an intermediate risk of developing CRC. That is based on the Government's acceptance in 2008 of the WHO cancer screening recommendations, including cost-effectiveness. Those that would fall within that classification have a familial RR of 3.15, with a range of 2.41 to 4.31 in the above table. Therefore, if the addition of societal and personal risk factors reaches the level that the government accepts as being eligible for a national program of targeted screening, then that should commence at the age of 50, or ten years younger than the youngest index case.

According to the present disclosure, entry into a screening program should then be for a period of no more than ten years. From that point, on the basis of the stratification study in this thesis, further surveillance should be based on the findings in those first ten years, or possibly a shorter period as the information accumulates and is analysed. As shown herein, an appropriate surrogate for CRC cancer risk is a group of polyps categorised as “Advanced Neoplasia” (AN). If there is no AN at the colonoscopy conducted at the age of 50 then it would be reasonable to delay the next colonoscopy until the age of 60. If at any point AN if found, then further surveillance should depend on that, according to well-established guidelines.

If at the end of the ten-year assessment period no AN have been found, the patient can be advised that the data indicate that while they are from a high-risk group, on the basis of their lifestyle and/or presumed genetic makeup, they are not personally at increased risk sufficient to warrant intensive surveillance. They could then be advised that appropriate lifestyle changes could reduce any risk, or that the presumed genetic contribution seems not to have been inherited.

If they have residual concerns because of previous advice, then an enhanced population screening program could be suggested, based on continuing developments in faecal testing. Such an AI-based system which relies on a relatively small number of quantitative parameters and presumably could be accommodated in a desktop module made available to family practitioners for example. All of the suggested parameters may be continually assessed and reassessed by an AI-based program.

At present, the definition of AN relates to two particular aspects. The first is the number of small non-significant adenomas that when found are categorised as AN, with numbers between 3 and 1 having been suggested. Some details are in the table below.

| | |
|---|---|
| Atkin Ref 368 | >1 small (<10 mm) TA RR 2.2 |
| de Jonge Ref 301 | >2 small TA RR 1.64 |
| Click Ref 605 | No difference between those |
| | with <3 NAA and >3 NAA |
| Dube Ref 371 | <3 small TA - no increased risk |
| He Ref 233 | <3 small TA - no increased risk |
| Kim Ref 809 | >2 small TA increased risk |
| Lieberman Ref 131 | >2 small TA RR 5.1 |
| Moon Ref 808 | >2 small TA RR 2.36 |
| Martinez Ref 240 | Small TA not regarded as AN |
| Pickhardt Ref 431 | Total volume rather than number |
| Sachdev Ref 326 | >2 small TA |
| Strum Ref 380 | >2 small AA increased risk |
| Taniguchi Ref 210 | >2 small AA increased risk |
| van Heijningen | 2 small AA RR 1.6; >2 RR 2.5 |
| Ref 304 | |
| van Stolk | >2 small AA increased risk, |
| Ref 700 | but also 2, although less so |

As well, the UK and USA definitions of AN differ in some aspects of histology, with one attributing a greater significance to a finding of villous pathology than the other. Such issues could be resolved with the accumulation of data over time.

With the increasing specificity of the recommendations, based on the accumulated data, the counselling of patients about the need or otherwise for screening colonoscopy could be delivered from an evidence-based source. This has been shown to enhance adherence to screening programs, the lack of which has been a constant factor contributing to lack of cost-effectiveness. It may also be one of the reasons that the possible decrease in incidence of a preventable cancer has not occurred.

In summary, a credible and effective method of stratifying individual patients for entry into screening programs and then for continuing participation is provided, on the results of the study disclosed herein, the information about risk factors that is now widely available, and the capacity of AI to interpret those data.

As disclosed above, the AI-Driven Algorithm harnesses deep learning. As such, the proposed solution will:
1. Model intricate non-linear interactions.
2. Seamlessly incorporate real-time patient data.
3. Consider a wide range of factors, from demographics to past medical conditions, to detailed imaging data.
4. Offer clinicians a personalised risk score for each colorectal cancer type and polyp, aiding in early interventions and better patient outcomes.

In one example, after 10 years of specialist consultations, a patient would receive a risk profile:
Risk of Cancer Type A in the subsequent decade: 80/100
Risk of Cancer Type B in the subsequent decade: 20/100

### Methodological Approach:

Supervised Learning: A substantial portion (70%) of the dataset, labeled with outcomes, is used for training, while the remaining 30% serves as a test bed to evaluate predictive accuracy.

Unsupervised Learning: The algorithm discerns underlying patterns and clusters within the data. These clusters are then mapped to significant predictors.

External Validation: To ensure the model's robustness and generalisability, it is be tested against an external dataset.

Continuous Learning with Deep Learning: In one example, the proposed method is a predominantly unsupervised deep learning model, capable of continuous learning and evolution as new data is introduced.

### Example Technical Infrastructure:

Hardware Requirements: A minimum of 16-32 GB RAM will be necessary for efficient data processing and model training.

Software & Integration: The disclosed methods may be implemented as an AWS/ML-compatible API. This API connects to a clinician-friendly portal where patient details can be input, and risk scores retrieved in real-time.

### Clinical Impact & Potential:

Early Detection & Intervention: With this algorithm, the medical community can potentially revolutionise early cancer detection. Clinicians, armed with a patient's medical history from the first 10 years of specialist check-ups, can determine risk scores, thereby guiding patients towards timely interventions, regular medical examinations, or even preventative procedures.

Cost-Efficiency: Earlier detections and interventions can lead to substantial reductions in healthcare costs, benefiting both patients and healthcare systems.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A method for detecting colorectal cancer in a subject, the method comprising:
determining whether an advanced neoplasm is present in a first screening time period; and
upon determining that an advanced neoplasm is present in the first screening time period, performing colonoscopy in a second screening time period, after the first screening time period, to detect colorectal cancer.

2. The method of claim 1, wherein the method further comprises upon determining that no advanced neoplasm is present in the first screening time period, performing community surveillance in the second screening time period.

3. The method of claim 2, wherein detection of no advanced neoplasm comprises detection of non-significant adenomas and no detection of advanced neoplasms.

4. The method of claim 1, wherein the first screening time period is 10 years.

5. A method for stratification of a subject, the method comprising:
upon detection of an advanced neoplasm in a first screening time period, stratifying the subject into a high risk stratification for developing colorectal cancer in a second screening time period.

6. The method of claim 5, wherein the method further comprises:
upon detection of no advanced neoplasm in the first screening time period, stratifying the subject into a low risk stratification for developing colorectal cancer in the second screening time period.

7. The method of claim 6, wherein detection of no advanced neoplasm comprises detection of non-significant adenomas and no detection of advanced neoplasms.

8. A computer implemented method for calculating a risk score for developing colorectal cancer, the method comprising:
determining patient data indicative of patient medical history and patient information;
applying a trained machine learning model to the patient data to determine the risk score; and
outputting the risk score.

9. The method of claim 8, wherein the patient medical history comprises results from a colonoscopy in a first screening time period.

10. The method of claim 9, wherein the results from the colonoscopy is indicative of advanced neoplasia in the first screening time period.

11. The method of claim 8, wherein the method further comprises selectively indicating a colonoscopy in a second screening time period based on the risk score.

12. The method of claim 8, wherein determining the patient data comprises extracting the patient data from patient medical records.

13. (canceled)

14. The method of claim 12, wherein the patient medical records are indicative of a history of polyps of types 1 to 5 in the first screening time period.

15. The method of claim 14, wherein the patient medical records further comprise one or more of:
demographics;
medical conditions other than colorectal cancer or polyps; and
medical imaging data.

16. The method of claim 8, wherein the trained machine learning model is a non-linear algorithm and the non-linear algorithm comprises one or more of:
a classification and regression tree;
a naive Bayes method;
a K-Nearest Neighbours method;
a Learning Vector Quantization method; or
a Support Vector Machine.

17. (canceled)

18. The method of claim 8, wherein the method further comprises training a machine learning model on historical patient data to obtain the trained machine learning model and the training comprises adjusting parameters of the machine learning model to reduce an error between (i) an output of the machine learning model and (ii) historical patient data indicative of a presence or absence of colorectal cancer associated with the historical patient data.

19. (canceled)

20. The method of claim 18, wherein the training comprises:
identifying clusters in the historical patient data; and
fitting the clusters to defining data points, and
the defining data points comprise a type of polyp detected in the first screening time period.

21. (canceled)

22. The method of claim 8, wherein the method further comprises:
determining a treatment plan based on the risk score; and
managing the patient by implementing the treatment plan.

23. The method of claim 22, wherein determining the treatment plan comprises determining a schedule of colonoscopy procedures based on the risk score.

24. The method of claim 8, wherein the patient medical data comprises data relating to lifestyle factors.
